(19) United States
(12) Patent Application Publication
(10) Pub. No.: US 2023/0347128 A1
(43) Pub. Date: Nov. 2, 2023

(54) Device for Prevention of Vascular Access Port Tampering

(71) Applicant: University of Rochester, Rochester, NY (US)

(72) Inventor: Kathleen M. Pratt, Rochester, NY (US)

(21) Appl. No.: 18/309,232

(22) Filed: Apr. 28, 2023

Related U.S. Application Data
(60) Provisional application No. 63/336,772, filed on Apr. 29, 2022.

(51) Int. Cl. A61M 39/10; A61M 39/16; A61M 39/20

(52) U.S. Cl. CPC A61M 39/1011; A61M 39/162; A61M 39/20

(57) ABSTRACT

The present disclosure relates to devices and methods for prevention of vascular access port tampering. In certain aspects, the devices and methods described herein protect a subject's vascular access from the transfer of extraluminal microorganisms and non-prescribed substances into the intraluminal aspect of their intravenous catheter and from invading their systemic blood stream.

### CROSS-REFERENCE TO RELATED APPLICATIONS

This application claims priority to U.S. Provisional Application No. 63/336,772, filed on Apr. 29, 2022, the disclosure of which is incorporated herein by reference in its entirety.

### BACKGROUND OF THE INVENTION

Bacteremia, blood stream infections (BSI), are significant and deadly, accounting for 150 deaths per 1000 patients affected with a Central Line Associated Blood Stream Infection (CLABSI) from central venous catheters (CVCs). Costs per patient for CLABSI are estimated to be from $27,232 to $68,989, depending on level of care and length of stay is estimated to increase an additional four to 22 days to recover from the infection (Alotaibi, Naif H., Abdulrahman Barri, and Muhammad A. Elahi. “Length of stay in patients with central line-associated bloodstream infection at a Tertiary Hospital in the Kingdom of Saudi Arabia.” Cureus 12.10 (2020); “Estimating the Additional Hospital Inpatient Cost and Mortality Associated With Selected Hospital-Acquired Conditions.” Agency for Healthcare Research and Quality, Rockville, MD., November 2017). In addition to CLABSI concerns, peripheral intravenous catheters (PIVC) have a failure rate of 90% from phlebitis and infection. An estimated 1 in 3 healthcare-associated BSIs are due to PIVCs (PIVC-BSI). Infections from PIVCs are considered preventable; however, incidence of PIVC-BSI among 85,063 PIVCs was 0.18% (midline catheters not included). With an estimated 200 million PIVCs placed annually in the United States and 2 billion placed worldwide, this equates to enormous potential for patient harm. In one study, the overall rate was 1.64 PIVC-BSIs per 1000 admissions, with 74.3% of the cases involving sepsis and 25.7% involving severe sepsis or septic shock (Nickel, Barb. “Hiding in plain sight: peripheral intravenous catheter infections.” Critical Care Nurse 40.5 (2020): 57-66). Although reported rates of PIVC-BSIs are lower than rates of CLABSIs, the exponentially higher use of PIVCs indicates that the absolute number of PIVC-BSIs is near to and may surpass the number of CLABSIs. Bacteremia can develop from peripheral vascular modalities that include phlebotomy punctures, PIVC lines, midlines, and low quality line maintenance. Blood stream infections from vascular access primarily occur due to failure in aseptic or sterile technique during catheter insertion, accessing the line, attaching to needless adapter, and maintenance of the venous access line.

Vascular access is an effective and efficient way to meet a patient's evolving health care needs while in an inpatient health care setting. Peripherally inserted central catheters (PICCs), CVCs, midlines, and PIVCs are devices commonly used by physicians to effectively administer fluids, medications, blood, blood products, and parenteral nutrition, or to obtain lab/phlebotomy needs for a patient on a routine basis throughout in-patient hospitalization (See FIGS. 1A-1C). The venous catheter is usually inserted in the hospital and some can remain in place for weeks or months. Although very convenient, the lengthy use of a PIVC and CVC increases the chances of developing bacteremia. Patients discharged with intravenous access PICC lines or CVCs, are a concern for self-harm if they are a patient who struggles with substance use disorder and use illicit substances intravenously. Unfortunately, ease of access can be a temptation to this population of patient. Self-administration of unprescribed quantities of illicit substances can possibly lead to overdose, blood stream infection, vascular damage, human immunodeficiency virus (HIV) infection, hepatitis, endocarditis, pulmonary, bone, and skin infections, psychiatric disorders (e.g., major depression, generalized anxiety disorder, posttraumatic stress disorder, personality disorders), violence, accidents, or death.

If a patient develops bacteremia due to PIVC or a CLABSI from a central line while in the hospital, the patient's length of stay in the hospital can, according to the Agency of Healthcare Research and Quality, result in excessive cost and with the additional required care, it can cost up to $160,000 per patient for treatment and medication with a septic infection (“Estimating the Additional Hospital Inpatient Cost and Mortality Associated With Selected Hospital-Acquired Conditions.” Agency for Healthcare Research and Quality, Rockville, MD., November 2017). Overall, hospital acquired infections (HAIs) cost at least $28.4 billion each year in the United States (Centers for Disease Control and Prevention. “Vital signs: central line—associated blood stream infections—United States, 2001, 2008, and 2009.” Annals of emergency medicine 58.5 (2011): 447-450). Under CLABSI, the source of the infection is attributed to the hospital, thus forcing the institution to pay for the hospital stay and extra medications used on the patient. However, if there is proof of tampering, the CLABSI may be downgraded to a Laboratory Confirmed Bloodstream Infection (LCBI) (For example, proof of tampering as provided by the PICC and Central Line Protection Clamp by Neuma Innovations). In this case, the healthcare system would no longer be responsible for the costs incurred for the secondary infection related to an extraluminal source of infection.

The need to actively protect lines and decrease chances of BSI related to PIVCs and CVCs is long overdue. Thus, there is a need in the art for a device to address tamper activity and BSI related to PIVCs and CVCs, as well secondary infection due to extraluminal vector sources causing infection. The present invention meets these needs.

### SUMMARY OF INVENTION

In one aspect, the present invention relates to a device for prevention of vascular access port tampering comprising: a housing unit having a proximal end, a distal end, a length therebetween, and an interior enclosure configured to receive a catheter line inlet port; an opening in the proximal end of the housing unit, passing through said housing unit to the interior enclosure and forming a peripheral rim on the proximal end of the housing unit configured to allow passage of tubes attached to the inlet port; and a plurality of tabs arranged circumferentially on the peripheral rim.

In one embodiment, the plurality of tabs are pointing radially inward and towards the distal end of the housing. In one embodiment, the plurality of tabs comprise one or more breakaway features for fracturing at least a portion of the plurality of tabs off of the peripheral rim. In one embodiment, the breakaway features are selected from the group consisting of: notches, indents, holes, perforations, reliefs and scores.

In one embodiment, the housing unit further comprises a cap positioned at the distal end within the interior enclosure, wherein the cap is configured to attach to the inlet port and block access to the catheter line. In one embodiment, the cap comprises a base and sidewalls extending up proximally from said base forming a cavity region with a rim.

In one embodiment, the device further comprises a sealing member connected to the rim and sealing the cavity region of the cap. In one embodiment, the sealing member is selected from the group consisting of: cellophane, PVC (Polyvinylidene Chloride), LDPE (Low-Density Polyethylene), polypropylene, foil, and antiseptic impregnated material.

In one embodiment, the device further comprises one or more antiseptic fluids held within the cavity region of the circular container. In one embodiment, the antiseptic fluids is selected from the group consisting of: isopropyl alcohol, isopropanol, chlorhexidine, povidone-iodine, chloroxylenol, hexachlorophene, benzalkonium chloride, and hydrogen peroxide.

In one aspect, the present invention relates to a device for prevention of vascular access port tampering comprising: a housing unit having a proximal end, a distal end and an interior enclosure configured to receive a catheter line inlet port thereinto; an opening in the proximal end of the housing unit, passing through said housing unit to the interior enclosure, and forming a peripheral rim on the proximal end of the housing unit configured to allow passage of tubes attached to the inlet port; and wherein the opening is formed with one or more separable proximal leaflets in the proximal end of the housing unit configurable from a first open position to a second closed position.

In one embodiment, the housing unit further comprises a cap positioned at the distal end within the interior enclosure, wherein the cap is configured to attach to the inlet port and block access to the catheter line. In one embodiment, the cap comprises a base and sidewalls extending up proximally from said base forming a cavity region with a rim.

In one embodiment, the device further comprises a sealing member connected to the rim and sealing the cavity region of the cap.

In one aspect, the present invention relates to a device for prevention of vascular access port tampering comprising: a housing unit having a proximal end, a distal end, a length therebetween, and an interior enclosure configured to receive a catheter line inlet port thereinto; and a sealing member positioned on the housing unit such that it would not permit access to the interior enclosure without distorting the device and giving evidence of tampering with the device.

In one embodiment, the housing unit comprises a first housing part, and a second housing part, wherein the first housing part engages with the second housing part to define the interior enclosure of the housing unit. In one embodiment, the first housing part and the second housing part are hingedly connected to each other at one end. In one embodiment, the first housing part and the second housing part comprise a locking mechanism at a second end selected from the group consisting of: a teeth locking mechanism, a tabbed locking mechanism, and a pencil-case type locking mechanism. In one embodiment, the housing unit further comprises a cap positioned at the proximal end within the interior enclosure, wherein the cap is configured to attach to the inlet port and block access to the catheter line.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIGS. 1A-1C depict vessel access locations and modalities on a subject. FIG. 1A depicts vessels and location of venous vascular access. FIG. 1B. depicts peripherally inserted central catheter (PICC) line insertion into a subject's arm. FIG. 1C depicts Central Venous Catheter (CVC) locations.

FIGS. 2A-2C depict an exemplary device of the present invention. FIG. 2A depicts a perspective view of an exemplary device according to aspects of the present invention. FIG. 2B depicts a perspective cutaway view (left) and bottom cutaway view (right) of an exemplary device. FIG. 2C shows exemplary dimensions for an embodiment of the device of FIG. 2A.

FIG. 3 is a flowchart depicting an exemplary method of using a device of the present invention.

FIGS. 4A and 4B depict an exemplary device of the present invention. FIG. 4A depicts a perspective view of an exemplary device according to aspects of the present invention. FIG. 4B shows exemplary dimensions for an embodiment of the device of FIG. 4A.

FIG. 5 is a flowchart depicting an exemplary method of using a device of the present invention.

FIG. 6 depicts a perspective view (left, middle) and a top down view (right) of an exemplary device of the present invention with a teeth locking mechanism.

FIG. 7 depicts a perspective view of an exemplary device of the present invention with a teeth locking mechanism.

FIG. 8 depicts step by step instructions for use of an exemplary device with a teeth locking mechanism. 1: Place the needleless connector (clave) in the device. Make sure that the tube is aligned at the bottom 2: Close the movable part to secure the device in place. 3: Remove tamper-evident seal to open 4: Open the device and take the clave out 5: Device is ready for use.

FIG. 9 depicts a perspective view of an exemplary device of the present invention with a pencil-case type locking mechanism.

FIG. 10 depicts step by step instructions for use of an exemplary device of the present invention with a pencil-case type locking mechanism. 1: Place the clave in the device. Make sure that the tube is aligned at the bottom 2: Close the movable part to secure the device in place 3: Remove tamper-evident seal to open 4: Open the device and take the clave out 5: Device is ready for use.

FIG. 11 depicts a perspective view of an exemplary device of the present invention with a sealing member as a closing mechanism.

FIG. 12 depicts step by step instructions for use of an exemplary device of the present invention with a sealing member as a closing mechanism. 1: Place the clave in the device. Make sure that the tube is aligned at the bottom 2: Close the movable part to secure the device in place Peel the seal to expose to sticky part of it and paste it over the closing mechanism 4: Remove the seal 5: Device is ready for use.

FIG. 13 is a flowchart depicting an exemplary method of using a device of the present invention.

FIG. 14 depicts design descriptions for exemplary devices of the present invention (design A and design B).

FIG. 15 depicts design descriptions for exemplary devices of the present invention (design C and design D).

FIG. 16 depicts design descriptions for exemplary devices of the present invention (design E, design F and design G).

FIG. 17 depicts design descriptions for exemplary devices of the present invention (design H and design I).

FIG. 18 depicts design descriptions for exemplary devices of the present invention (design J, design K and design L).

FIG. 19 depicts a design for manufacturing (DFM) configuration for the printing of part A: The top left is the top view, the top right is the isometric view, the bottom right is the right view, and the bottom left is the front view. All the measurements are in mm.

FIG. 20 depicts a design for manufacturing (DFM) configuration for the printing of part B: The top left is the top view, the top right is the isometric view, the bottom right is the right view, and the bottom left is the front view. All the measurements are in mm.

FIG. 21 depicts a design for manufacturing (DFM) configuration for the printing of part C: The top left is the top view, the top right is the isometric view, the bottom right is the right view, and the bottom left is the front view. All the measurements are in mm.

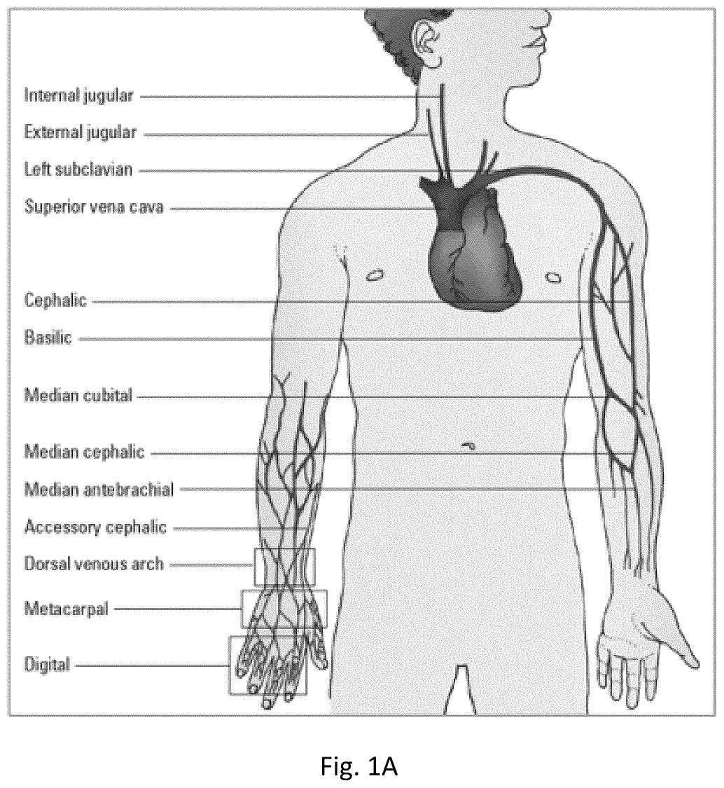

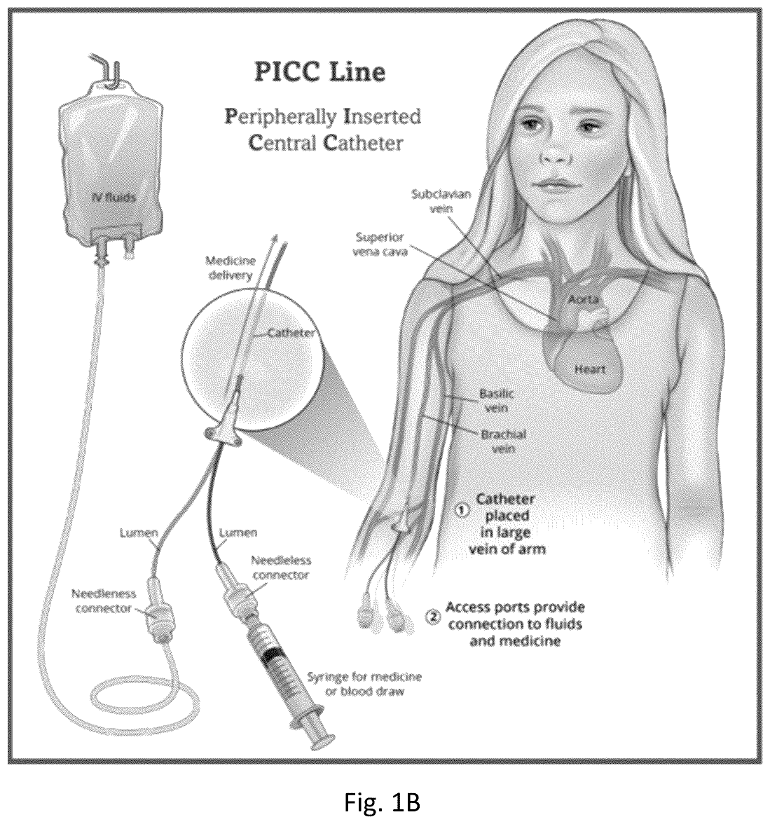

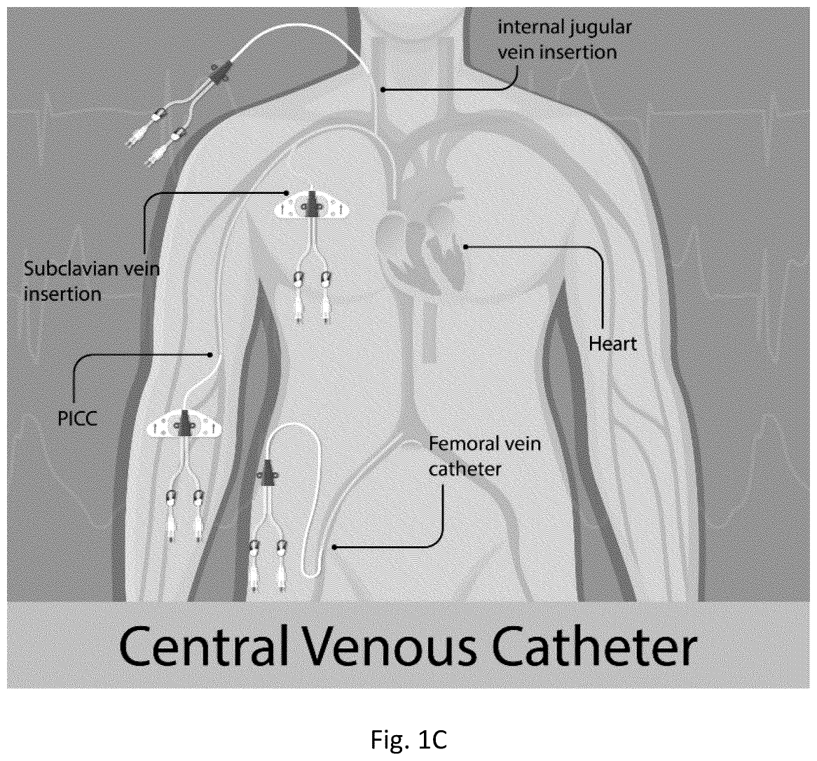

### DETAILED DESCRIPTION

It is to be understood that the figures and descriptions of the present invention have been simplified to illustrate elements that are relevant for a clear understanding of the present invention, while eliminating, for the purpose of clarity many other elements found in the field of catheter injection port lock. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The disclosed device was developed out of an identified need to protect patients and their vascular access from extraluminal sources of infection or fomites.

The disclosed device has the potential to positively impact health care outcomes for patient and clinician. The disclosed device can improve the healthcare experience, quality of care, reduce patient length of stay, and reduce the financial healthcare burden of secondary infections (CLABSI and PIVC-BSI) and their sequalae. Vascular access modalities to be utilized with the disclosed device include but are not limited to: PIVCs, midlines, CVCs (including internal jugular (IJ) and femoral lines), tunneled lines, PICCs, or Implanted venous access devices (IVADs).

In some embodiments, the disclosed device is a small plastic medical device designed to offer tamper evident protection for needleless connectors intended for use with intravenous therapy. In some embodiments, the disclosed device is a single use, break-away design that cannot be reused, that offers evidence of removal and provides awareness that the line may not be clean, may have been tampered with, or is compromised. In some embodiments, the disclosed device protects a subject's vascular access from the transfer of extraluminal microorganisms and non-prescribed substances into the intraluminal aspect of their intravenous catheter and from invading their systemic blood stream.

In some embodiments, the disclosed device is a form fitted cover that surrounds the catheter's needleless connector and houses a built in antiseptic impregnated material that passively disinfects the needleless connector as it remains in place and in contact with the needleless connector.

### Catheter Port Lock Device

The present invention provides tamper evident vascular access devices configured to deter or prevent a subject, such as an intravenous drug user (IVDU), from tampering with their vascular access. For example, the presently described devices can be used for deterring self-injecting and self-inflicted harm, preventing prolonged hospital stays, preventing incurred secondary infections, preventing comorbidities/sequalae development, preventing alternate/prolonged treatment trajectory or complications to treatment course, and preventing poor health outcomes.

In some embodiments, the devices of the present invention are configured to protect all types of regularly used vascular access lines where subjects would not be under constant supervision. These devices include, but are not limited to, a peripheral cannula (PC), a short midline, a midline, a central venous catheter (CVC), an acute non-tunneled CVC (ANT-CVC), peripherally inserted central catheter (PICC) lines, tunneled lines, implanted venous access device (IVADS), internal jugular central venous lines (IJ's), Peripheral internal venous (IV), fem lines, midlines, US guided IV, etc. In some embodiments, the devices of the present invention are configured to keep these lines protected and clean and decrease the occurrence of bloodline infections. In some embodiments, the devices of the present invention are used by the nurses and bedside hospital staff responsible for placing and removing the device and the patients on whom it would be placed. In some embodiments, the devices of the present invention may be used for any suitable candidate known to one skilled in the art including but not limited to children, patients with neurodegenerative diseases, psychiatric patients, intravenous drug users, illicit drug users, animals, etc.

### Catheter Port Lock Device—Push On Style

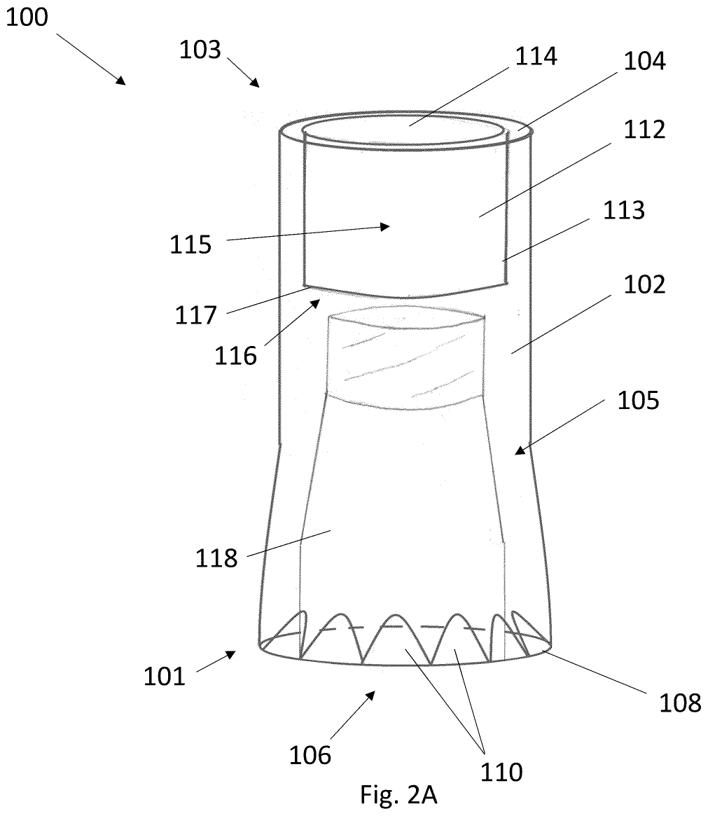

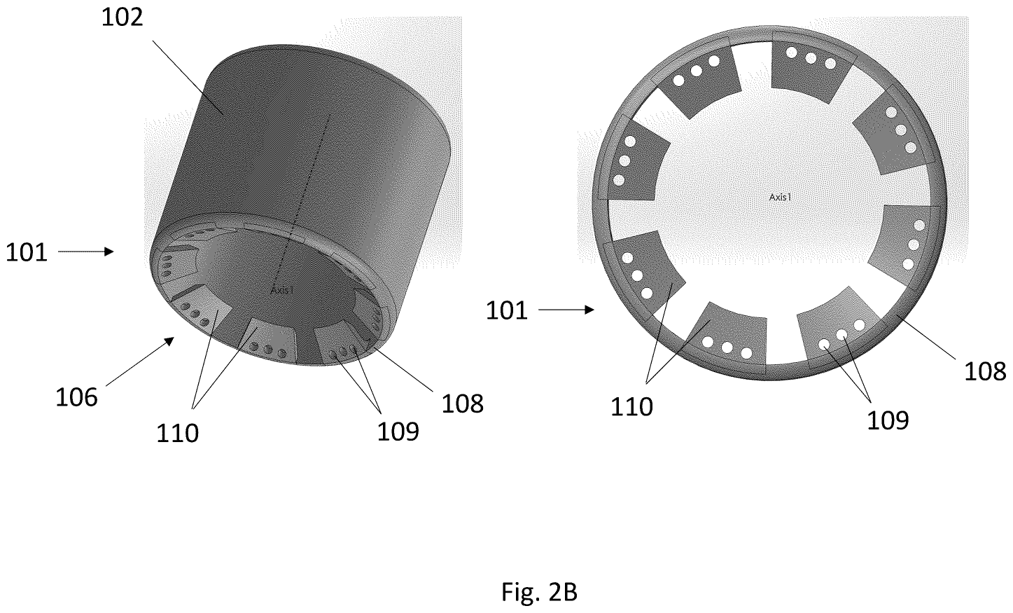

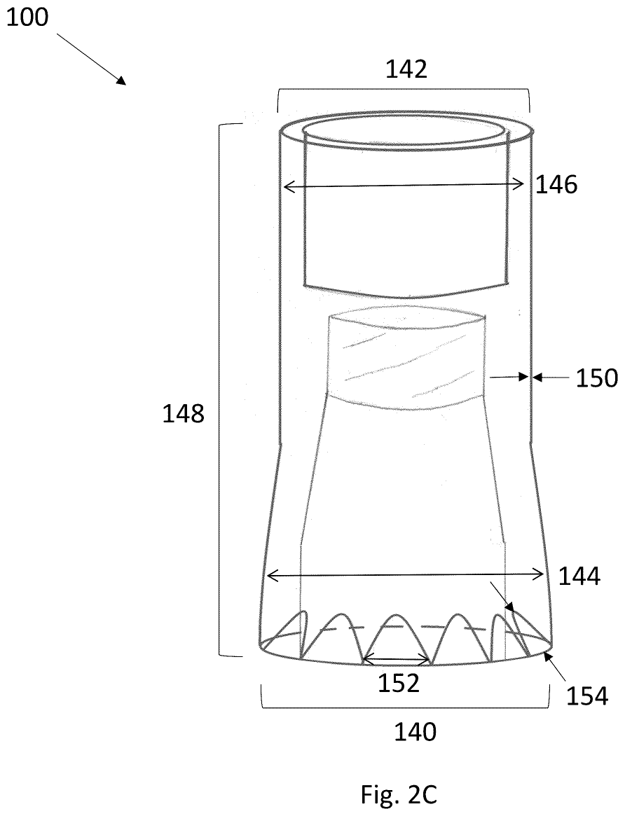

In some aspects, the present invention relates to a push-on style catheter port lock device. Referring now to FIG. 2A, shown is an exemplary catheter port lock device 100 comprising a housing 102 having a proximal end 101, a distal end 103 and a length therebetween. In some embodiments, housing 102 is a cylindrical container comprising a distal bottom 104 and a proximal opening 106 forming an interior region 105. In some embodiments, opening 106 is substantially circular and comprises a proximal peripheral rim 108. In some embodiments, a plurality of tabs 110 are attached to and extend out from peripheral rim 108 and protrude into the interior of housing 102. In some embodiments, plurality of tabs 110 are arranged circumferentially around peripheral rim 108. In some embodiments, plurality of tabs 110 extend inward radially from peripheral rim 108 and point in a distal direction. In some embodiments, plurality of tabs 110 allow a needleless connector 118 to pass through opening 106 of housing 102 in a distal direction, and fixedly position needleless connector 118 within interior region 105. In some embodiments, plurality of tabs 110 are “breakaway” features that snap or break when needleless connector 118 is removed in a proximal direction out from opening 106. In some embodiments, plurality of tabs 110 are a tamper-evident feature, wherein one or more broken tabs indicates device 100 was used, tampered with, and/or a needless connector 118 was removed.

In some embodiments, device 100 further comprises a tamper-evident strap attached to a portion of housing 102 proximate to the peripheral rim 108. In some embodiments, the strap is integral to a portion of housing 102. In some embodiments, the tamper-evident strap is configured to secured device 100 to needless connector 118. In some embodiments, the tamper-evident strap includes a suitable closure apparatus for closing the tamper-evident strap upon itself when the strap is folded around the device 100 and needless connector 118. In some embodiments the strap includes a tamper evident feature that is designed for a single use, and is designed to break when opened to show evidence of tampering.

In some embodiments, device 100 further comprises a cap 112 having a base 114 and a circular sidewall 113 extending upward from the base and forming a cavity 115 with an opening 116. In some embodiments, opening 116 comprises a peripheral rim 117 whereon one or more sealing members and/or sealing layers may be affixed. In some embodiments, the one or more sealing members is an antiseptic impregnated film. In some embodiments, cap 112 forms a single unit with housing 102, and is positioned within interior region 105 of housing 102 near the distal end. In some embodiments, the base 114 and bottom 104 comprise a shared surface, and the circular sidewall 113 of cap 112 extends in a proximal direction from bottom 104 of housing 102. In some embodiments, cap 112 is a separate unit from housing 102, and may be removably positioned in the interior region 105 of housing 102. In some embodiments, cap 112 may be fixedly and removably attached to a needless connector 118.

In some embodiments, cap 112 provides an antiseptic barrier to prevent pathogens, microorganisms, or the like from entering the vascular access port. In some embodiments, cap 112 is an antiseptic cap, wherein cavity 115 is held in sterile conditions until device 100 and/or cap 112 are used. In some embodiments, cap 112 further comprises a sealing member, sealing layer, and/or layer of material attached to peripheral rim 117 of circular sidewalls 113, wherein the sealing member encloses cavity 115. In some embodiments, the sealing member enclosing cavity 115 is ruptured when a needleless connector 118 extends through opening 116 of cap 112. In some embodiments, the sealing member is an anti-septic impregnated material that passively disinfects at least a portion of needleless connector 118 while the device is in use. In some embodiments, cavity 115 comprises one or more antiseptic solutions contained and sealed within cavity 115 of cap 112, sealed in the cavity with the one or more sealing members.

Referring now to FIG. 2B, shown is a perspective (left) and bottom (right) cutaway view of an exemplary proximal end 101 for device 100 showing opening 106 and plurality of tabs 110. Plurality of tabs 110 are configured to allow a needleless connector 118, and the like, to pass through opening 106 in a proximal direction without breaking the tabs, however, if the needleless connector is pulled out in a distal direction, one or more tabs will break and/or deform. The removal of the needless connector 118 and the breaking of one or more tabs of plurality of tabs 110 may indicate that device 100 has been tampered with and/or used. In some embodiments, plurality of tabs 110 comprise one or more breakaway features 109 in plurality of tabs 110. In some embodiments, breakaway features 109 are one or more holes passing through plurality of tabs 110. In some embodiments, breakaway features 109 are cut-out reliefs in plurality of tabs 110. In some embodiments, breakaway features 109 are thinner or thicker regions of material along the height or width of plurality of tabs 110. In some embodiments, breakaway features may comprise notches, indents, holes, perforations, reliefs and/or scores.

Referring now to FIG. 2C, shown is an exemplary device 100 with labelled dimensions. In some embodiments, housing 102 is sized to create a snug fit for a port, connector, clave (e.g. needleless connector 118), PICC line assembly, and the like, so that the connector does not have the space to rotate and/or become dislodged, and is held fixedly and removably in place within interior region 105.

In some embodiments, housing 102 has a proximal outer diameter 140 of about 15-30 mm. In some embodiments, housing 102 may have a proximal outer diameter 140 of more than 30 mm. In some embodiments, housing 102 may have a proximal outer diameter 140 of less than 15 mm.

In some embodiments, housing 102 has a distal outer diameter 142 of about 15-30 mm. In some embodiments, housing 102 may have a distal outer diameter 142 of more than 300 mm. In some embodiments, housing 102 may have a distal outer diameter 142 of less than 15 mm.

In some embodiments, housing 102 may have a proximal inner diameter 144 of about 15-25 mm. In some embodiments, housing 102 may have a proximal inner diameter 144 of more than 25 mm. In some embodiments, housing 102 may have a proximal inner diameter 144 of less than 15 mm.

In some embodiments, housing 102 may have a distal inner diameter 146 of about 15-25 mm. In some embodiments, housing 102 may have a distal inner diameter 146 of more than 25 mm. In some embodiments, housing 102 may have a distal inner diameter 146 of less than 15 mm.

In some embodiments, housing 102 may have a length 148 of about 50-100 mm. In some embodiments, housing 102 may have a length 148 of more than 100 mm. In some embodiments, housing 102 may have a length 148 of less than 50 mm.

In some embodiments, housing 102 may have a wall thickness 150 of about 0.5 mm-3 mm. In some embodiments, housing 102 may have a wall thickness 150 of more than 3 mm. In some embodiments, housing 102 may have a wall thickness 150 of less than 0.5 mm. In some embodiments, wall thickness 150 is variable along length 148 of device 100. For example, in some embodiments, wall thickness 150 is thicker in a distal portion of housing 102, and thinner in a proximal portion of housing 102, or vice versa.

In some embodiments, plurality of tabs 118 have a width 152 and a height 156. In some embodiments, plurality of tabs 118 have a width 152 of approximately 0.1 25 mm to 5 mm. In some embodiments, plurality of tabs 118 have a width of about 3 mm. In some embodiments, plurality of tabs 118 have a height 154 of approximately 0.1 mm to 6 mm. In some embodiments, plurality of tabs 118 have a height of about 3 mm.

Aspects of the present invention relate to one or more materials for any device of the present invention (i.e. device 100, and device 300 and 500 as described below). In some embodiments, device 100, 200 and/or 300 comprise polycarbonate, metal, titanium, aluminum, steel, alloy, polymer, PLA, PVA, PEEK, ABS, PTFE and/or PP. In some embodiments, device 100, 200 and/or 300 comprises a waterproof material, a water-resistant material, a non-porous material, a low porous material, an antimicrobial material, a low pore material, an impermeable material. In some embodiments, device 100, 200 and/or 300 comprises cellophane, foil, PVC (Polyvinylidene Chloride), LDPE (Low-Density Polyethylene), polypropylene, a three-layer laminate composed of a layer of meltblown polypropylene bonded on both surfaces with a layer of spunbonded polypropylene. In some embodiments, device 100, 200 and/or 300 comprises alcohol, isoproplyl alcohol, propanol, antiseptic liquids, and other antiseptic solutions as would be known by one of ordinary level of skill in the art. In some embodiments, device 100, 200 and/or 300 comprises an antimicrobial coating, a waterproof coating, a water-resistant coating, a hydrophobic coating.

### Method of Use—Push On Style

The present invention also relates to methods for locking access to catheter inlet ports to prevent or deter a subject, such as an intravenous drug user (IVDU) from tampering with their vascular access. In certain embodiments, the method of present invention is used for deterring self-injecting and self-inflicted harm, preventing prolonged hospital stays, preventing incurred secondary infections, preventing comorbidities/sequalae development, preventing alternate/prolonged treatment trajectory or complications to treatment course, and preventing poor health outcomes. In some embodiments, the method of present invention is configured to keep these lines protected and clean and decrease the occurrence of bloodline infections.

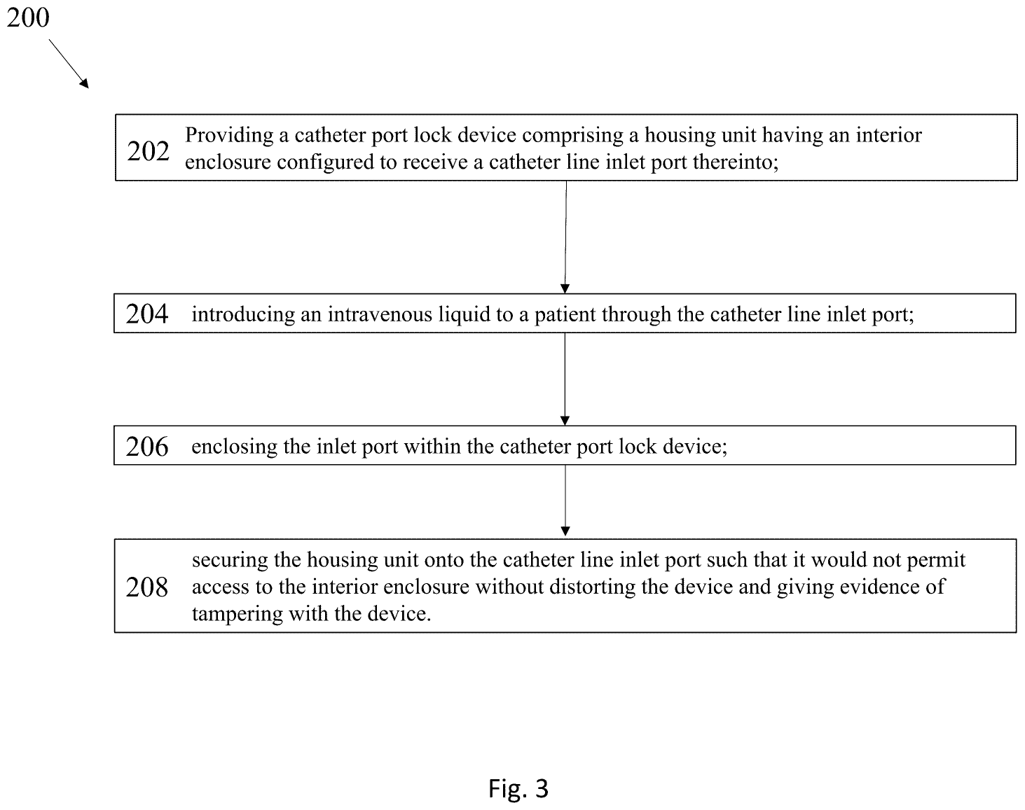

Referring now to FIG. 3, an exemplary method 200 of administering intravenous liquids to a patient is depicted. In some embodiments, the method allows for locking access to the inlet port to protect the intravenous drug use (IVDU) patient from tampering with their vascular access and to reduce chances of developing bloodline infections. Method 200 begins with step 202, wherein a catheter port lock device comprising a housing unit having an interior enclosure configured to receive a catheter line inlet port thereinto is provided. In step 204, an intravenous liquid is introduced to a patient through the catheter line inlet port. In step 206, the inlet port is enclosed within the catheter port lock device. In step 208, the housing unit is secured onto the catheter line inlet port such that it would not permit access to the interior enclosure without distorting the device and giving evidence of tampering with the device. In certain embodiments, the inlet port is enclosed within the catheter port lock device and the housing unit is secured without prior administration or introduction of an intravenous liquid through the catheter line port.

### Catheter Port Lock Device—Clam Style

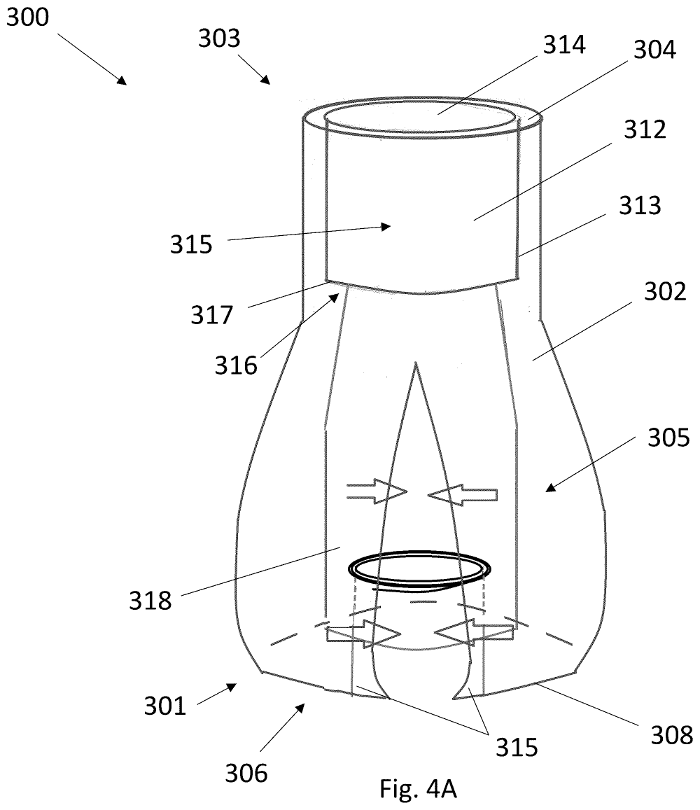

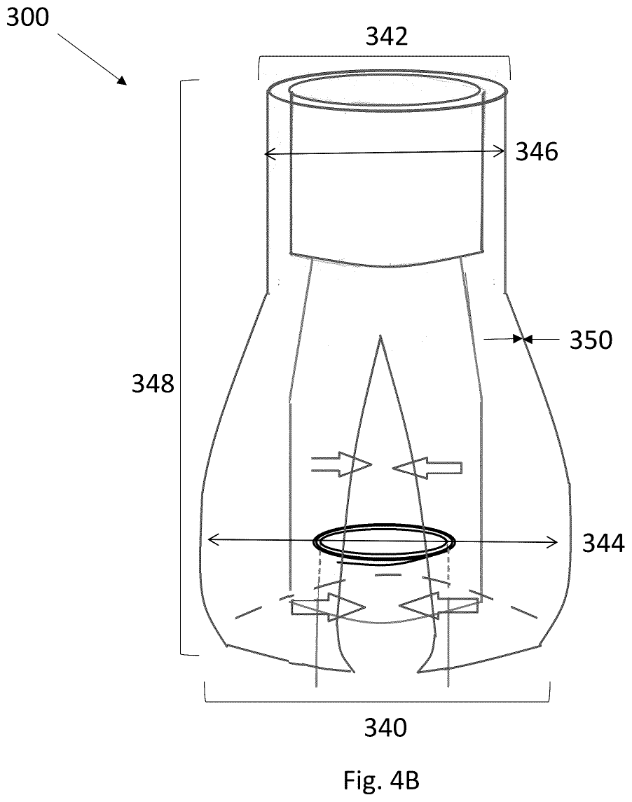

Aspects of the present invention relate to a clam style catheter port lock device. Referring now to FIG. 4A, shown is an exemplary catheter port lock device 300 comprising a housing 302 having a proximal end 301, a distal end 303 and a length therebetween. In some embodiments, housing 302 is a cylindrical container comprising a distal bottom 304 and proximal opening 306 forming an interior region 305. In some embodiments, the proximal end of housing 302 comprises an opening 306 formed by a plurality of leaflets 315. In some embodiments, plurality of leaflets 315 form a “clam-shell” design that may open or close between a first open position, and a second closed position. In some embodiments, plurality of leaflet 315, when open in the first position allow a needless connector 318 to pass through opening 306 of housing 302 in a distal direction and position needless connector 318 within interior region 305. In some embodiments, when plurality of leaflets 315 are closed in the second position, opening 306 is substantially smaller, circular and comprises a continuous proximal peripheral rim 308. In some embodiments, plurality of leaflets 315, when in the closed second position, prevents needleless connector 318 from being removed in a proximal direction and/or tampered with. In some embodiments, plurality of leaflets further comprise a tamper-evident feature, such as a temper-evident tape or securement, wherein one or more tamper-evident indicators on the tamper-evident feature will indicate if needleless connector 318 was removed and/or plurality of leaflets 315 are opened to the first position from the second closed position.

In some embodiments, device 300 further comprises a cap 312 having a base 314 and a circular sidewall 313 extending upward from the base and forming a cavity 315 with an opening 316. In some embodiments, opening 316 comprises a peripheral rim 317 whereon one or more sealing members and/or sealing layers may be affixed. In some embodiments, cap 312 forms a single unit with housing 302, and is positioned within interior region 305 of housing 302 near the distal end. In some embodiments, the circular sidewall 313 of cap 312 extends in a proximal direction from bottom 304 of housing 302. In some embodiments, cap 312 is a separate unit from housing 302, and may be removably positioned in the interior region 305 of housing 302. In some embodiments, cap 312 may be fixedly and removably attached to a needless connector 318.

In certain embodiments, cap 312 provides an antiseptic barrier to prevent pathogens, microorganisms, or the like from entering the vascular access port. In some embodiments, cap 312 is an antiseptic cap, wherein cavity 315 is held in sterile conditions until device 300 and/or cap 312 are used. In some embodiments, cap 312 further comprises a sealing member, sealing layer, and/or layer of material attached to peripheral rim 317 of circular sidewalls 313, wherein the sealing member encloses cavity 315. In some embodiments, the sealing member enclosing cavity 315 is ruptured when a needleless connector 318 extends through opening 316 of cap 312. In some embodiments, cavity 315 comprises one or more antiseptic solutions sealed within cavity 315 of cap 312 with the one or more sealing members or layers.

Referring now to FIG. 4B, shown is an exemplary device 300 with labelled dimensions. In some embodiments, housing 302 is sized to create a snug fit for a port, connector, clave (e.g. needleless connector 318), PICC line assembly, and the like, so that the connector does not have the space to rotate and become dislodged.

In some embodiments, housing 302 has a proximal outer diameter 340 of about 15-30 mm. In some embodiments, housing 302 may have a proximal outer diameter 340 of more than 30 mm. In some embodiments, housing 302 may have a proximal outer diameter 340 of less than 15 mm.

In some embodiments, housing 302 has a distal outer diameter 342 of about 15-30 mm. In some embodiments, housing 302 may have a distal outer diameter 342 of more than 30 mm. In some embodiments, housing 302 may have a distal outer diameter 342 of less than 15 mm.

In some embodiments, housing 302 may have a proximal inner diameter 344 of about 15-20 mm. In some embodiments, housing 302 may have a proximal inner diameter 344 of less than 15 mm. In some embodiments, housing 302 may have a proximal inner diameter 344 of more than 20 mm.

In some embodiments, housing 302 may have a distal inner diameter 346 of about 15-20 mm. In some embodiments, housing 302 may have a distal inner diameter 346 of less than 15 mm. In some embodiments, housing 302 may have a distal inner diameter 346 of more than 20 mm.

In some embodiments, housing 302 may have a length 348 of about 50-100 mm. In some embodiments, housing 302 may have a length 348 of more than 100 mm. In some embodiments, housing 302 may have a length 348 of less than 50 mm.

In some embodiments, housing 302 may have a wall thickness 350 of approximately 0.1 mm-3 mm. In some embodiments, housing 302 may have a wall thickness 350 of more than 3 mm. In some embodiments, housing 302 may have a wall thickness 350 of more than 0.1 mm. In some embodiments, wall thickness 350 is variable along length 348 of device 300. For example, in some embodiments, wall thickness 350 is thicker in a distal portion of housing 302, and thinner in a proximal portion of housing 302, or vice versa.

### Method of Use—Clam Style

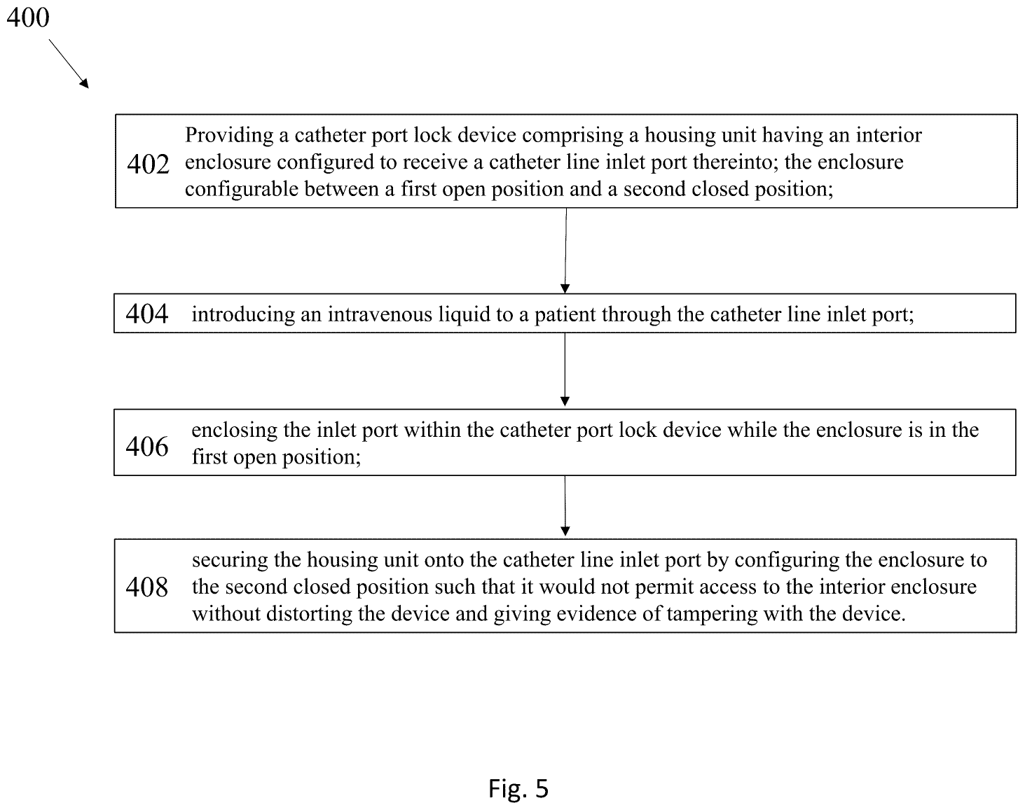

Referring now to FIG. 5, an exemplary method 400 of administering intravenous liquids to a patient is depicted. In some embodiments, the method allows for locking access to the inlet port to protect the intravenous drug use (IVDU) patient from tampering with their vascular access and to reduce chances of developing bloodline infections. Method 400 begins with step 402, wherein a catheter port lock device comprising a housing unit having an interior enclosure configured to receive a catheter line inlet port thereinto and the enclosure is configurable between a first open position and a second closed position is provided. In step 404, an intravenous liquid is introduced to a patient through the catheter line inlet port. In step 406, the inlet port is enclosed within the catheter port lock device while the enclosure is in the first open position. In step 408, the housing unit is secured onto the catheter line inlet port by configuring the enclosure to the second closed position such that it would not permit access to the interior enclosure without distorting the device and giving evidence of tampering with the device. In certain embodiments, the inlet port is enclosed within the catheter port lock device and the housing unit is secured without prior administration or introduction of an intravenous liquid through the catheter line port.

### Catheter Port Lock Device—Hinged Style

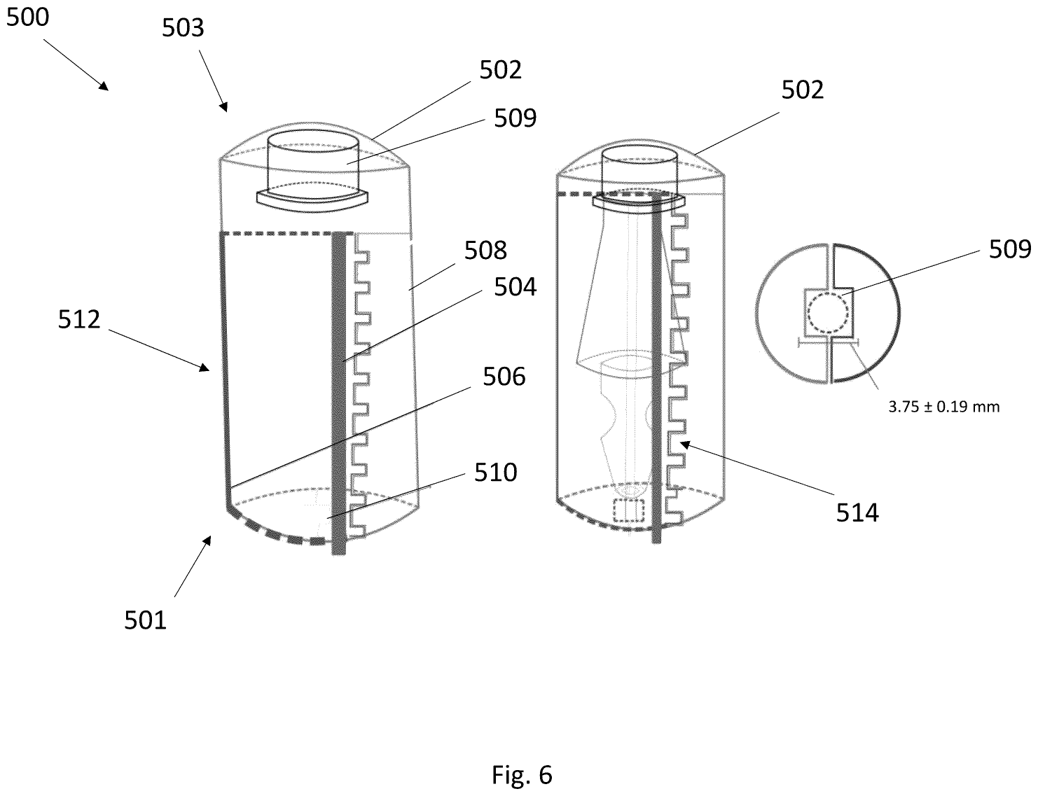

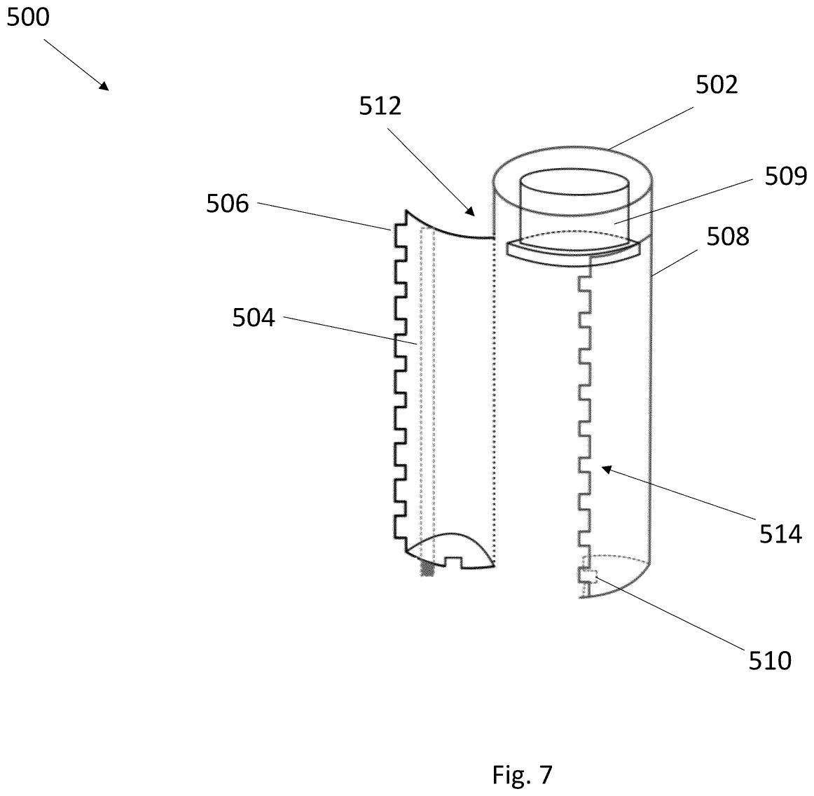

Referring now to FIG. 6 and FIG. 7, an exemplary catheter port lock device 500 of the present invention is shown. Device 500 comprises a housing unit 502 and a sealing member 504 attached thereto. In some embodiments, housing unit 502 is a hollow tubular structure having a proximal end 501, a distal end 503, and comprises a first housing part 506, a second housing part 508 and, in some examples, a cap 509. In some embodiments, first and second housing parts 506 and 508 may be made with any suitable method known to one skilled in the art including but not limited to 3D printing, injection molding, etc. First and second housing parts 506 and 508 have cooperating structures which allow the parts to slide and/or hingedly attach together to form housing unit 502 and define a housing compartment of adequate dimensions to receive an inlet port and any associated connector including but not limited to a needleless connector (clave) of a vascular access line (e.g., PICC) and to protect the port from access by the patient. In some embodiments, first and second housing parts 506 and 508 may be the same size. In some embodiments, first and second housing parts 506 and 508 may be differently sized with one being larger than the other.

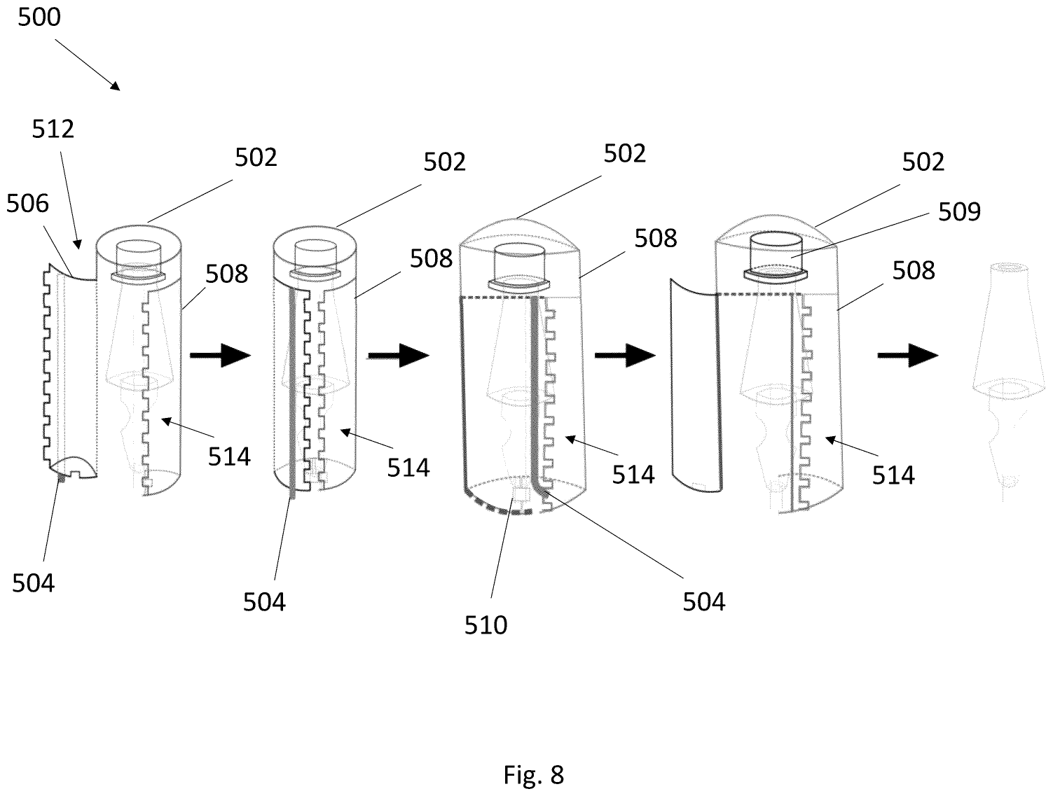

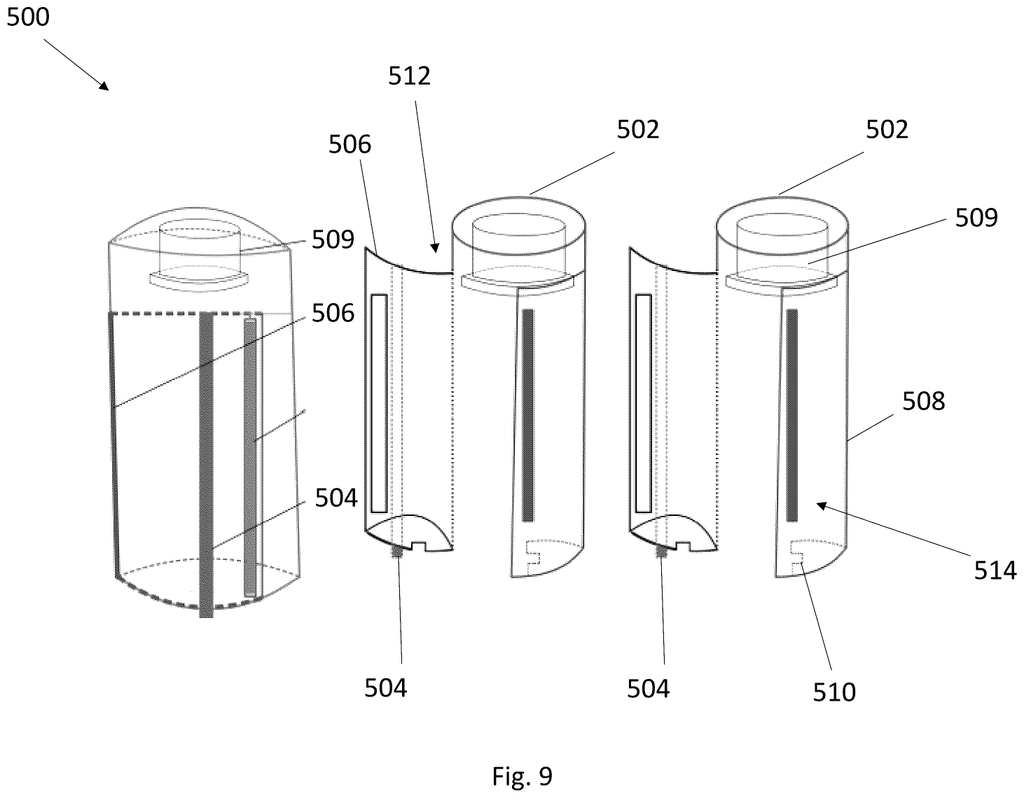

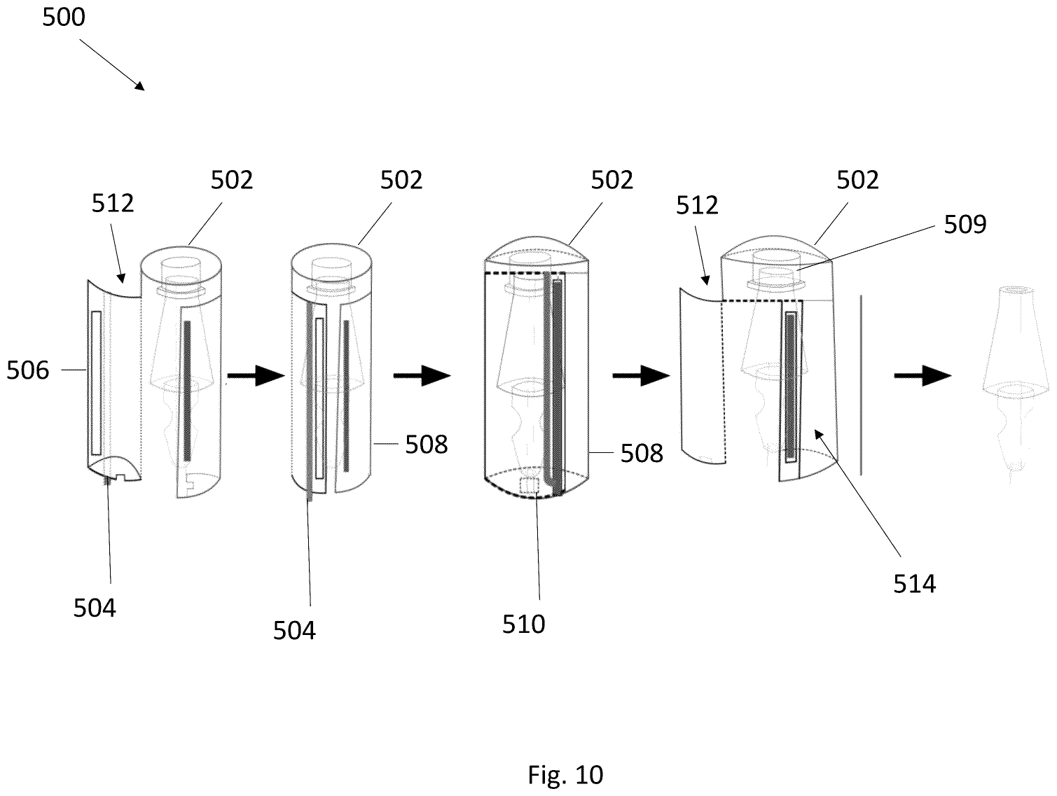

First and second housing parts 506 and 508 when joined together form a first opening 510, configured to allow passage of tubing from the connector. First opening 510 may have any suitable shape known to one skilled in the art including but not limited to circular. In some embodiments, first and second housing parts 506 and 508 may be hingedly connected to each other at a first end 512. In some embodiments, first and second housing parts 506 and 508 may be fixedly, sealingly and/or removably joined together at a second end 514 with any mechanism known to one skilled in the art including but not limited to a teeth locking mechanism (FIG. 6, FIG. 7 and FIG. 8), a pencil-case type locking mechanism (FIG. 9 and FIG. 10), a snap-fit, a compression fit, a tongue and groove, a detent, etc.

In some embodiments, housing unit 502 is dimensioned to create a snug fit for the PICC line assembly comprised of the needleless connector (clave), so that the needleless connector does not have the space to rotate and become dislodged. In some embodiments, housing unit 502 has an outer diameter of about 19-21 mm. In some embodiments, housing unit 502 may have an outer diameter of more than 21 mm. In some embodiments, housing unit 502 may have an outer diameter of less than 19 mm. In some embodiments, housing unit 502 may have an inner diameter of about 16.15-17.85 mm. In some embodiments, housing unit 502 may have an inner diameter of more than 17.85 mm. In some embodiments, housing unit 502 may have an outer diameter of less than 16.15 mm. In some embodiments, housing unit 502 may have a length of about 61.75-68.25 mm. In some embodiments, housing unit 502 may have a length of more than 68.25 mm. In some embodiments, housing unit 502 may have a length of less than 61.75 mm. In some embodiments, housing unit 502 may have a wall thickness of approximately 1.42-1.58 mm. In some embodiments, housing unit 502 may have a wall thickness of more than 1.58 mm. In some embodiments, housing unit 502 may have a wall thickness of more than 1.42 mm.

Cap 509 is positioned at proximal end of housing 502 across from first opening 510 and is configured to engage the needless connector and to block access into the interior of the needleless connector (clave), while housing unit 502 is in place. In certain embodiments, cap 509 comprises internal threads to engage threads of the needless connector (clave). In certain embodiments, cap 509 provides an antiseptic barrier to prevent pathogens, microorganisms, or the like from entering the vascular access port.

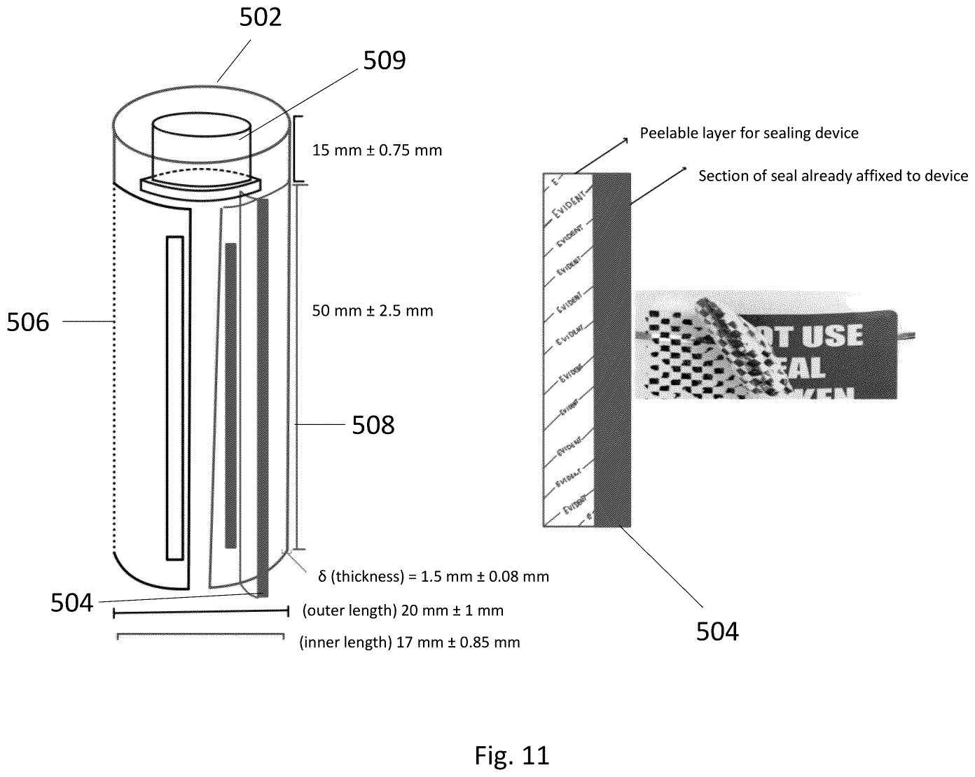

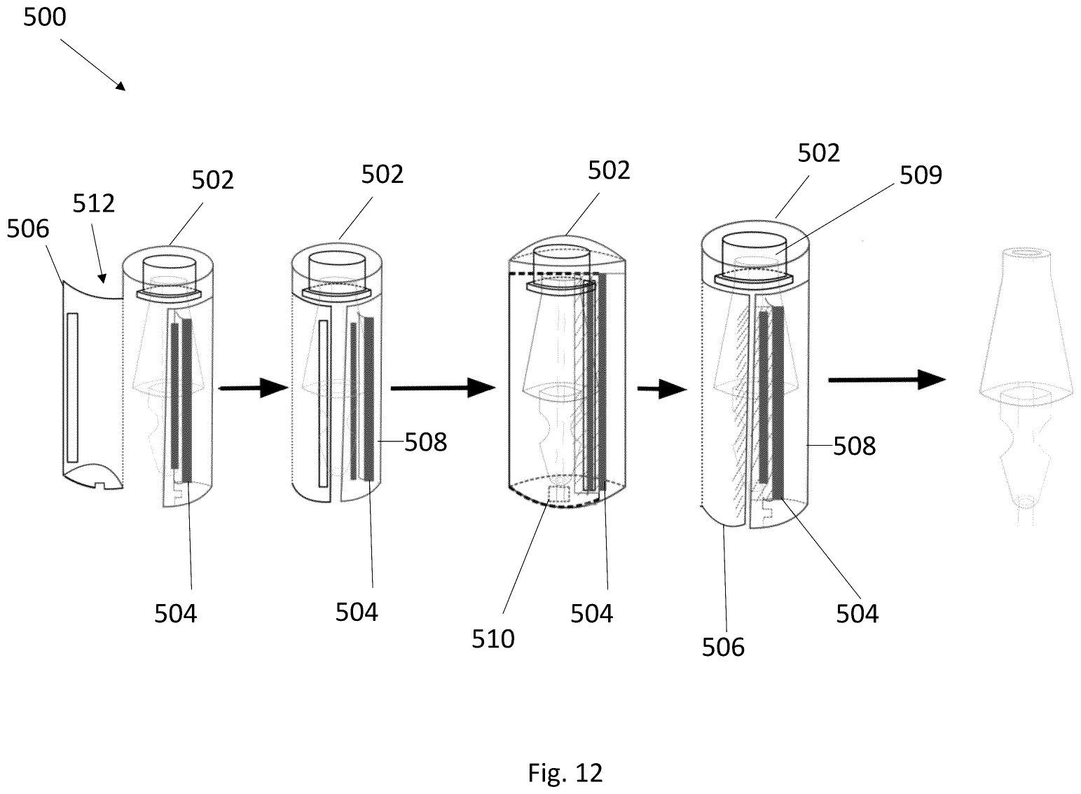

In some embodiments, sealing member 504 is positioned anywhere on device 500 such that first and second housing parts would not be able to separate without removing sealing member 504. In some embodiments, sealing member may be positioned top of second housing part 508. In some embodiments, sealing member 504 may be positioned on top of both first and second housing parts 506 and 508. In some embodiments, sealing member 504 may be used to lock first and second housing parts 506 and 508 together (FIG. 11 and FIG. 12). In this embodiment, sealing member 504 may comprise a section that is already attached to one of first and second housing parts 506 and 508, wherein the other section can be peeled and pasted over the seal.

In some embodiments, any device of the present invention (e.g. device 100, 300, and 500) may be used in any temperature ranging between −10-130° F. In some embodiments, any device of the present invention (e.g. device 100, 300, and 500) may be adjusted to adapt to all central catheters and IV lines.

In some embodiment, any device of the present invention (e.g. device 100, 300, and 500) is a single use device. In some embodiments, any device of the present invention (e.g. device 100, 300, and 500) may be stored and packaged in sterile conditions for about 2 years.

In one aspect, the present invention provides a system or kit comprising any disclosed device (e.g. device 100, 300, 500), as described herein, and one or more additional components or devices for providing vascular access in a subject. In some embodiments, the system or kit comprises a peripheral cannula (PC), a short midline, a midline, an acute non-tunneled CVC (ANT-CVC), peripherally inserted central catheter (PICC) lines, tunneled lines, implanted venous access device (IVADS), internal jugular central venous lines (IJ's), Peripheral internal venous (IV), fem lines, midlines, US guided IV, etc. in some embodiments, the system or kit comprises a needless connector.

### Method of Use—Hinged Device

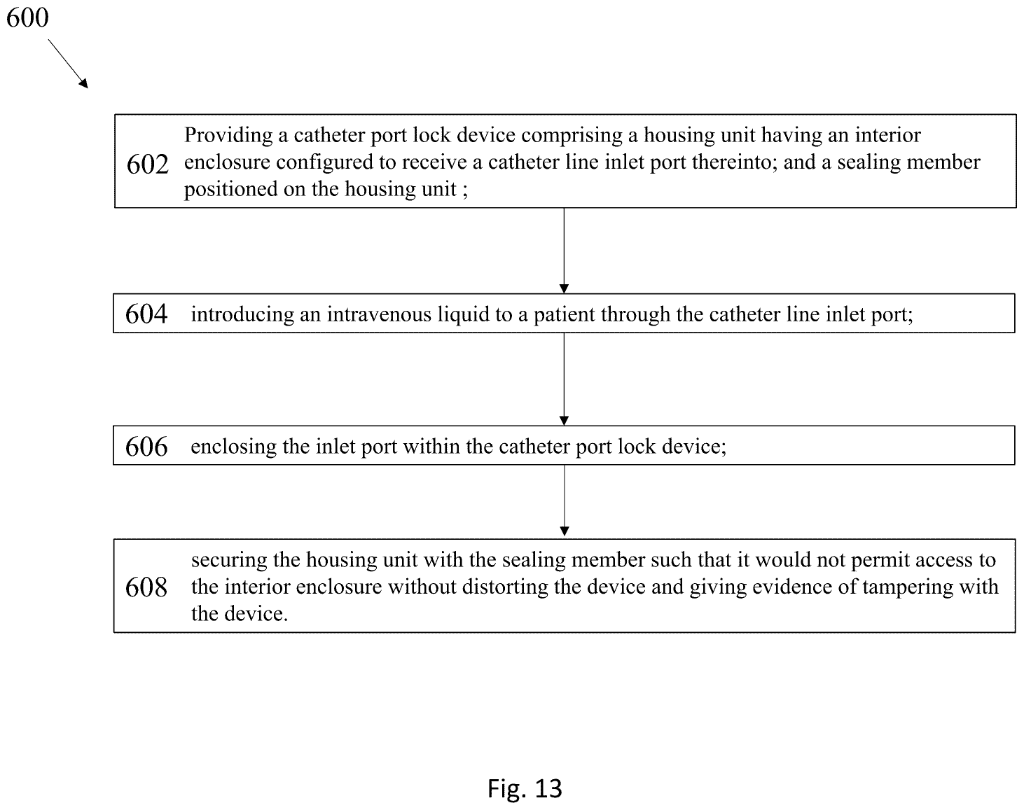

Referring now to FIG. 13, an exemplary method 600 of administering intravenous liquids to a patient is depicted. In some embodiments, the method allows for locking access to the inlet port to protect the intravenous drug use (IVDU) patient from tampering with their vascular access and to reduce chances of developing bloodline infections. Method 600 begins with step 602, wherein a catheter port lock device comprising a housing unit having an interior enclosure configured to receive a catheter line inlet port thereinto; and a sealing member positioned on the housing unit is provided. In step 604, an intravenous liquid is introduced to a patient through the catheter line inlet port. In step 606, the inlet port is enclosed within the catheter port lock device. In step 608, the housing unit is secured with the sealing member such that it would not permit access to the interior enclosure without distorting the device and giving evidence of tampering with the device. In certain embodiments, the inlet port is enclosed within the catheter port lock device and the housing unit is secured without prior administration or introduction of an intravenous liquid through the catheter line port.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out exemplary embodiments of the present invention, and are not to be construed as limiting in any way the remainder of the disclosure.

### Example 1: Design Variations and Selection Criteria

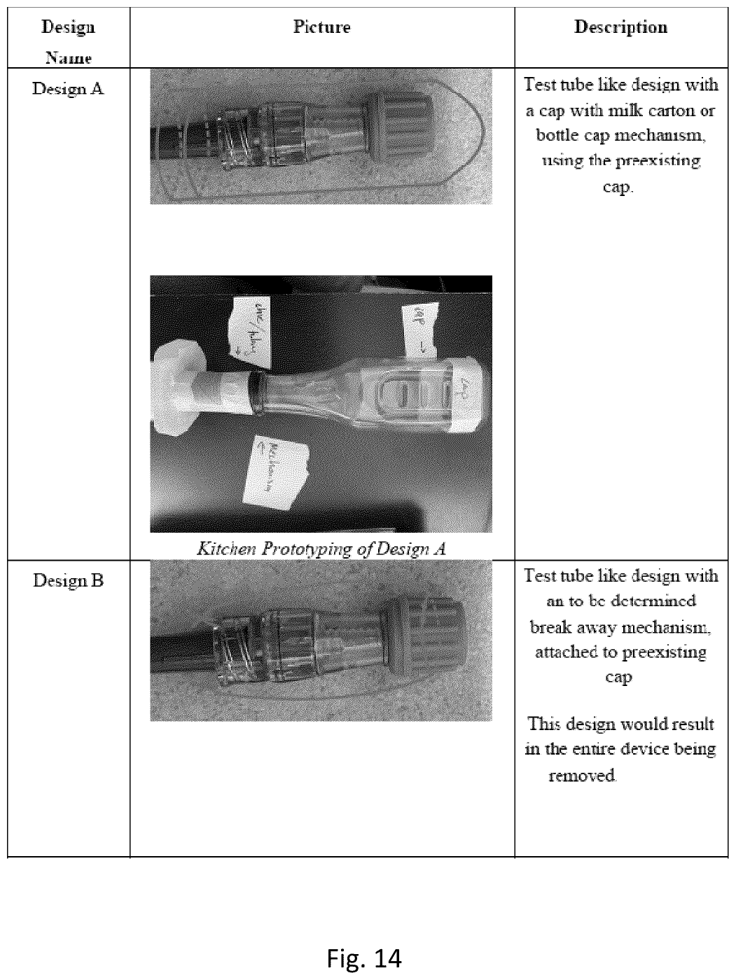

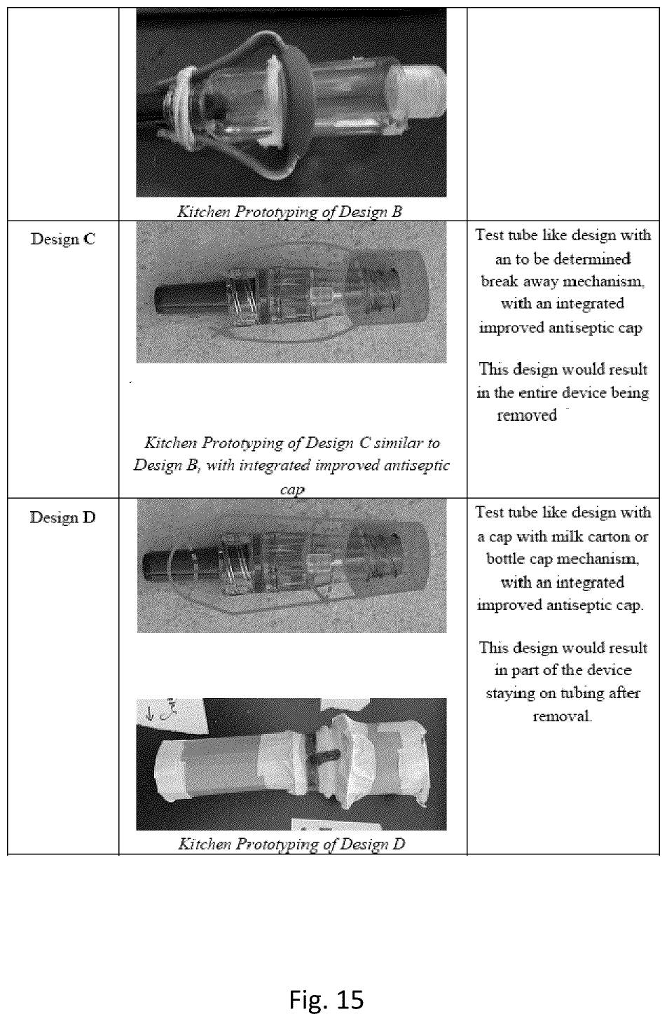

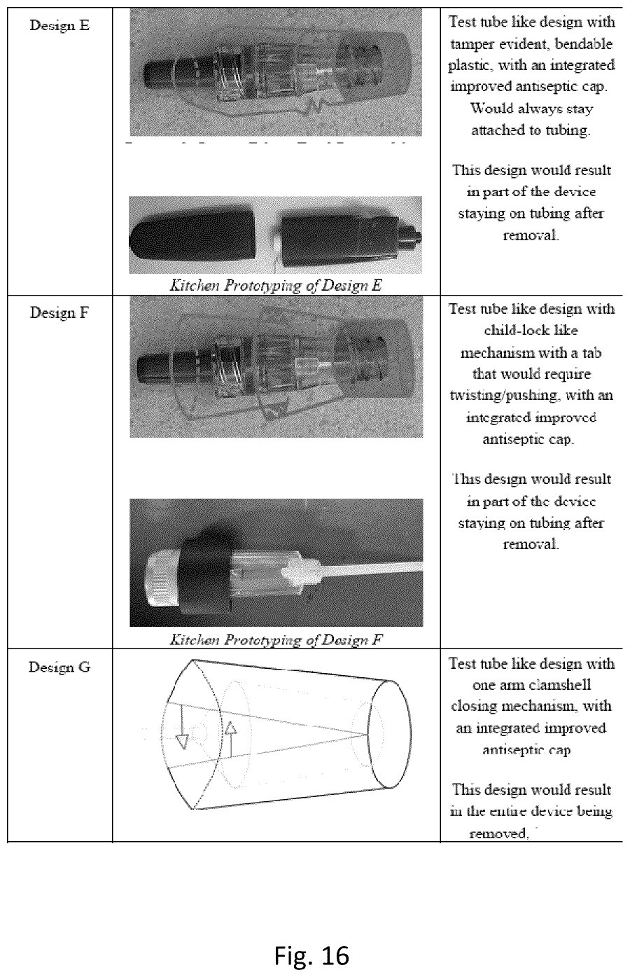

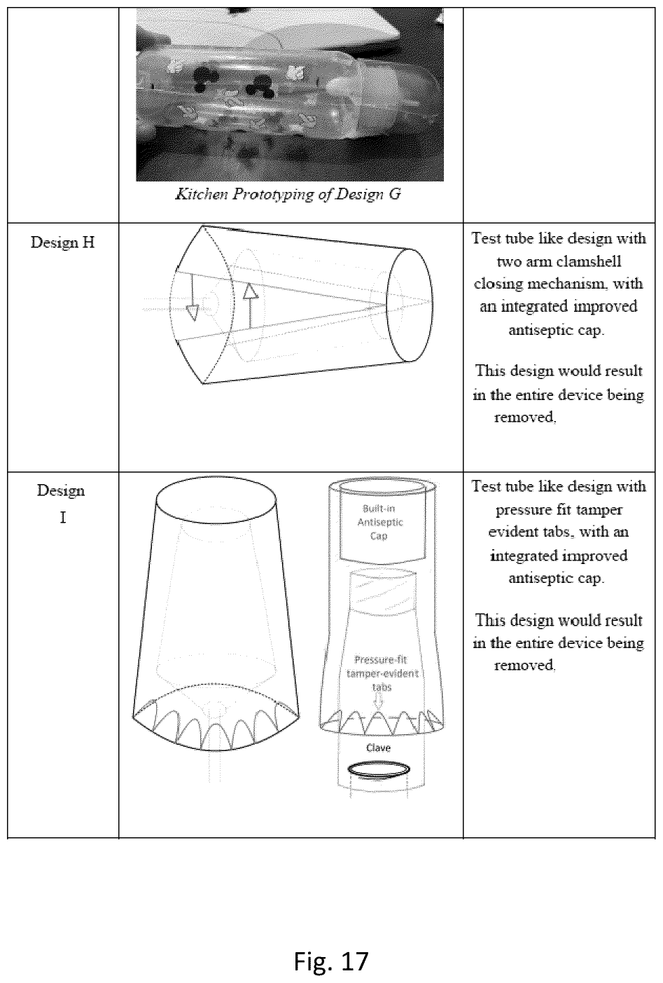

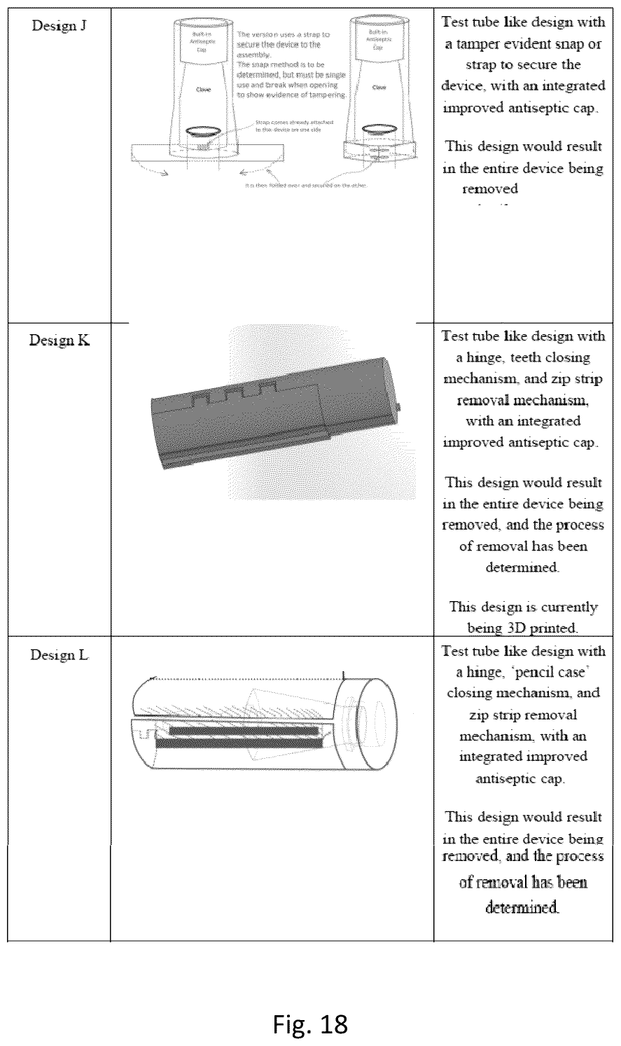

Several different design ideas with different locking mechanism were investigated (FIG. 14 through FIG. 18).

Design Selection

The requirements (Selection Criteria) most valued by customers that are relevant to the selection process were used to compare all prototyped designs (FIG. 14 through FIG. 18) against each other. The selection criteria in Table 1 includes the requirement, a description, how the requirement would be rated, and the corresponding test that was done or will be performed. Some of the tests, such as ‘Tamper Evident’ are based on the Functional Testing performed on the existing tamper-evident device, PICC Guard, in their 510(k)-application summary (PICC Guard LLC).

The requirements noted with an asterisk (Nurse Flow, Universal Product, Affordable) have yet to be determined due to lack of quantifiable data. The assigned values are based on the complexity of the design and anticipated expectations of the designs relative to each other. Although scored by all members as an attempt to be objective, true testing of the viable designs yield objective results. The testing performed to quantify these requirements are in Table 2. For example, for affordability the cost is based on general prices of similar items/materials, along with the research and understanding of various manufacturing options. The existing volume of the CAD prototype was used to estimate the price knowing the correlation between volume and cost, as well as online estimates from manufacturing companies. For nurse flow learning time, the column is left blank intentionally since objectively rating of this criterion was not done.

TABLE 1
Description of Selection Criteria

| Rank | Requirement | Description | Requirement | Testing |
|---|---|---|---|---|
| 0 | Protects clave | Ability to withstand | y/n | Mechanical testing to |
| | | 70 MPa of pressure | | measure the force required |
| | | (Omnexus) to access | | to break the device |
| | | clave inside device | | |
| 1 | Antiseptic Cap | Ability to incorporate | y/n | Physically testing if |
| | | an antiseptic cap into | | the existing cap can |
| | | the design | | fit inside the device |
| 2a | Tamper evident- | Can visibility indicate | y/n | Sample testing if subjects |
| | Visual Indicator | that the device has | | can tell the difference |
| | | been tampered with | | before and after tampering, |
| | | | | if tampered devices [symbol] |
| 2b | Tamper evident- | Includes inherent way | y/n | Sample testing where |
| | Irreversible | for device to not be | | subjects attempt to |
| | | able to put back | | reverse the tampering |
| | | together | | and test if other subjects [symbol] |
| 3a | Infection | Body can be disinfected | y/n | Material testing of body |
| | Prevention- | | | of device (w/o cap) for |
| | Disinfect | | | disinfection |
| 3b | Infection | Process for device to be | y/n | Sample testing if subjects |
| | Prevention- | entirely removed without | | can apply and remove the |
| | Removable | removing clave determined | | device without removing |
| | | | | the clave |
| 4a | *Nurse Flow- | Time to apply device | 3: <30 sec | Sample testing to measure |
| | Application | once taught | 2: 30 sec-1 min | average time it takes to |
| | Time | | 1: >1 min | apply the device once taught |
| | | | 0: Unknown | |
| 4b | *Nurse Flow- | Time to remove device | 3: <10 sec | Sample testing to measure |
| | Removal Time | once taught | 2: 10-20 sec | average time it takes to |
| | | | 1: >20 sec | remove device once taught |
| | | | 0: Unknown | |
| 4c | *Nurse Flow- | Time to initially learn | 3: <10 min | Sample testing to measure |
| | Learning Time | application of device | 2: 10-20 min | average time it takes to |
| | | | 1: >20 min | understand mechanism |
| | | | 0: Unknown | |
| 5 | *Universal | Ability to minimally | 3: All central line | Physically testing if |
| | product | adjust dimensions to | catheters + IV lines | other lines can fit in |
| | | adapt to other lines | 2: All central line catheters | existing dimensions of |
| | | | 1: PICC lines only | device, and/or attempting |
| | | | 0: Unknown | to alter CAD drawings to |
| | | | | fit other lines if needed |
| 6 | *Affordable | Ability for device to | 3: $0-5 | Sending prototype design |
| | | be produced at a similar | 2: $5-20 | to be manufactured to get |
| | | to rate to similar | 1: $21> | cost |
| | | products on the market | 0: Unknown | |
| Feasibility | Manufacturability | Ability to be manufactured | P: Able to be 3D printed | Research and discussions |
| | | | IM: Able to be injection molded | with experts in these |
| | | | O: Other method needed | manufacturing methods |

[symbol]

indicates data missing or illegible when filed

Using the above selection criteria from Table 1, all designs shown in FIG. 14 through FIG. 18 were scored in Table 2 below. As noted above, the requirements noted with an asterisk (Nurse Flow, Universal Product, Affordable) have yet to be determined due to lack of quantifiable data. The results of this matrix is used to determine which ideas to further develop and design to allow for further testing.

TABLE 2
Selection Matrix for Previous Prototyped Ideas

| | | Designs | | | | | | | | | | | |
|---|---|---|---|---|---|---|---|---|---|---|---|---|---|
| Rank | Requirement | A | B | C | D | E | F | G | H | I | J | K | L |
| 0 | Protects clave | Y | Y | Y | Y | Y | Y | Y | Y | Y | Y | Y | Y |
| 1 | Antiseptic Cap | N | N | Y | Y | Y | Y | Y | Y | Y | Y | Y | Y |
| 2a | Tamper evident- | Y | Y | Y | Y | Y | Y | N | N | Y | Y | Y | Y |
| | Visual Indicator | | | | | | | | | | | | |
| 2b | Tamper evident- | Y | Y | Y | Y | Y | N | N | N | Y | Y | Y | Y |
| | Irreversible | | | | | | | | | | | | |
| 3a | Infection Prevention- | Y | Y | Y | Y | Y | Y | Y | Y | Y | Y | Y | Y |
| | Disinfect | | | | | | | | | | | | |
| 3b | Infection Prevention- | N | Y | Y | N | N | N | Y | Y | Y | N | Y | Y |
| | Removable | | | | | | | | | | | | |
| 4a | *Nurse Flow- | 2 | 2 | 2 | 2 | 1 | 1 | 2 | 2 | 1 | 1 | 2 | 2 |
| | Application Time | | | | | | | | | | | | |
| 4b | *Nurse Flow- | 1 | 1 | 1 | 1 | 1 | 1 | 2 | 2 | 1 | 2 | 2 | 2 |
| | Removal Time | | | | | | | | | | | | |
| 4c | *Nurse Flow- | 0 | 0 | 0 | 0 | 0 | 0 | 0 | 0 | 0 | 0 | 0 | 0 |
| | Learning Time | | | | | | | | | | | | |
| 5 | *Universal product | 3 | 2 | 2 | 3 | 3 | 3 | 3 | 3 | 3 | 3 | 3 | 3 |
| 6 | *Affordable | 3 | 2 | 2 | 3 | 3 | 3 | 3 | 3 | 2 | 3 | 3 | 3 |

Table 3 summarizes the feasibility of all the designs regarding manufacturing. For the rating row, P=able to be 3D printed, IM=able to be injection molded, 0=other method needed. For the comments row, 1=Breakaway piece included, which presents challenges in terms of nurse flow and patient safety, 2=adhesive/tape included in design, which was unwanted by the customer in relevance to nurse workflow, 3=very thin (<1 mm) part needed in design, which would provide challenges with low resolution 3D printers, 4=conversations and research about 3D printing raises challenges with producing design, and 5—Bendable parts included in design, which requires careful material selection.

TABLE 3
Feasibility of designs

| | Designs | | | | | | | | | | | |
|---|---|---|---|---|---|---|---|---|---|---|---|---|
| | A | B | C | D | E | F | G | H | I | J | K | L |
| | Rating | | | | | | | | | | | |
| | | | | | | | | | | P | | |
| | P | IM | IM | IM | IM | P | IM | IM | | IM | P | P |
| | IM | O | O | O | O | IM | O | O | O | O | IM | IM |
| Comments/ | | 1 | 1 | | 5 | | 5 | 5 | 1 | 2 | 3 | 3 |
| Concerns | | | | | | | 4 | 4 | 4 | | | |

3D Printing of Designs

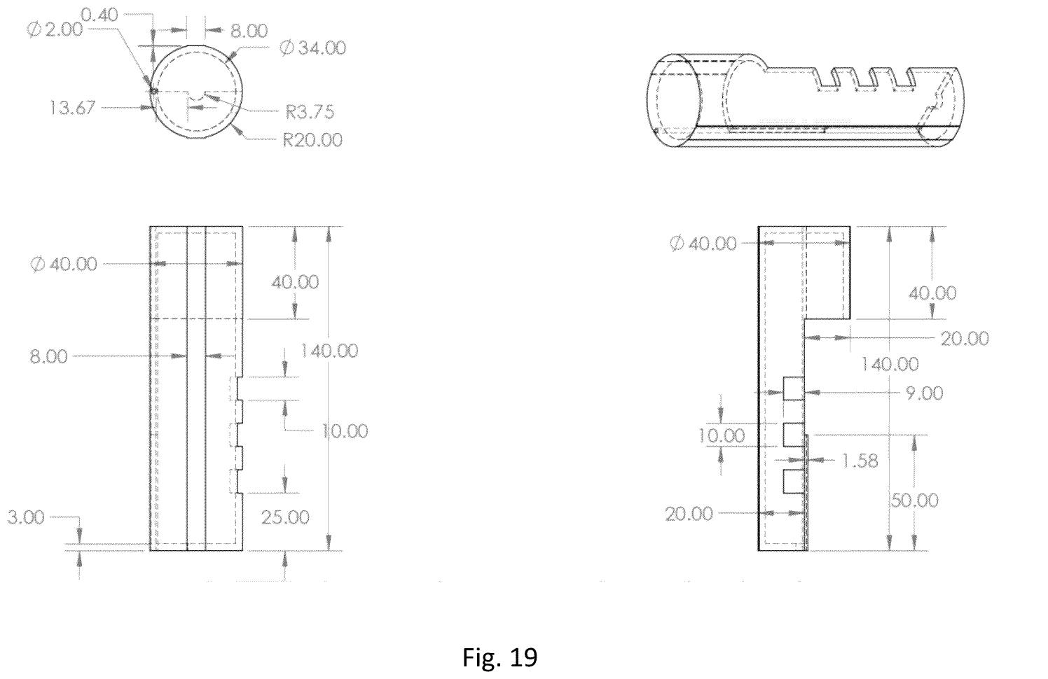

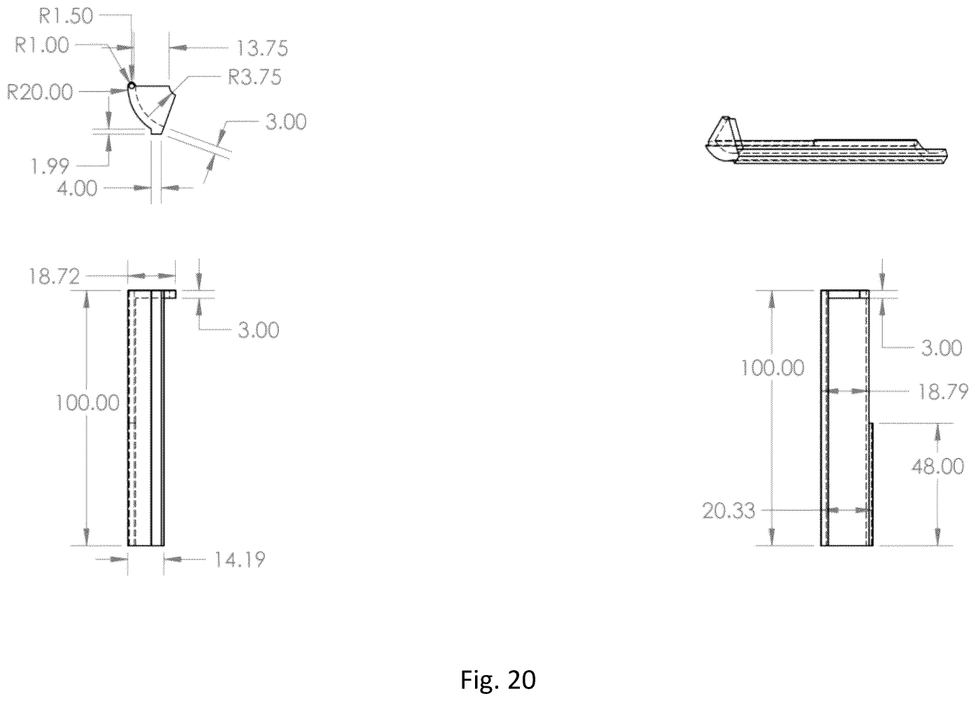

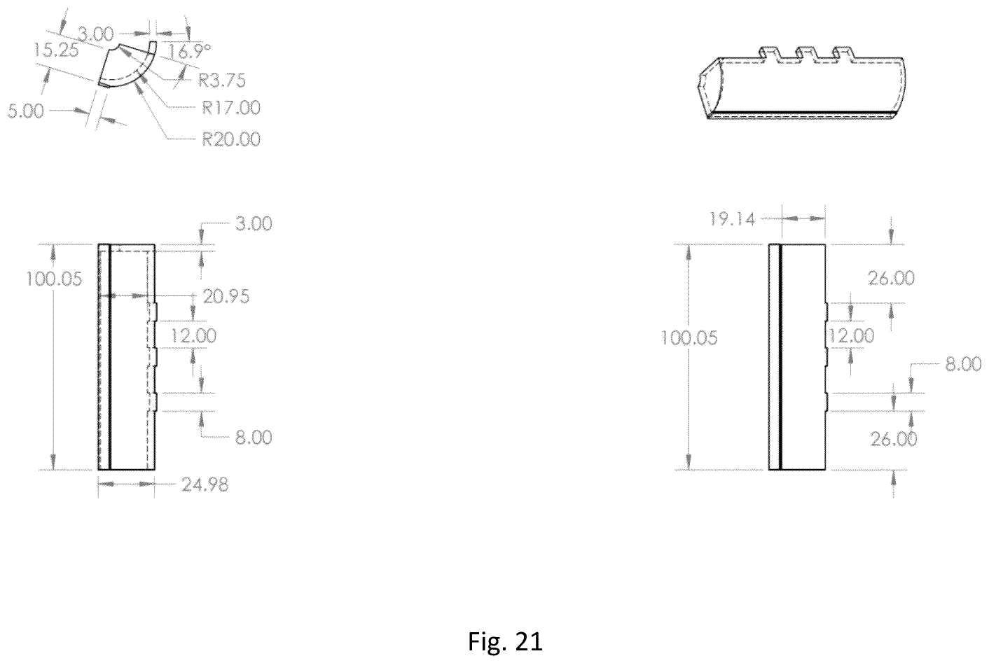

To ensure that the small features, like the teeth/the pencil case-mechanism bar and the hinge fit together, they need to be printed parallel to the printing surface. A flat surface has been added to the subparts of each prototype to help it print properly. FIG. 19 through FIG. 21 depicts the drawings of the three subparts submitted for printing. The first is part A, the largest part in the build. It has the space where the cap goes at the top and at the bottom one side has the teeth and the other has half of the hinge. The second part is part B. It is one of the two sides of the bottom attachment. On the longer sides of this part, one side has the space for the rip strip, and the other side has the second half of the hinge. The third part is part C. It is the other half of the bottom attachment. One the longer sides of this part, one side has the space for the rip strip, and the other side has the teeth.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A device for prevention of vascular access port tampering comprising:
a housing unit having a proximal end, a distal end, a length therebetween, and an interior enclosure configured to receive a catheter line inlet port;
an opening in the proximal end of the housing unit, passing through said housing unit to the interior enclosure and forming a peripheral rim on the proximal end of the housing unit configured to allow passage of tubes attached to the inlet port; and
a plurality of tabs arranged circumferentially on the peripheral rim.

2. The device of claim 1, wherein the plurality of tabs are pointing radially inward and towards the distal end of the housing.

3. The device of claim 2, wherein the plurality of tabs comprise one or more breakaway features for fracturing at least a portion of the plurality of tabs off of the peripheral rim.

4. The device of claim 3, wherein the breakaway features are selected from the group consisting of: notches, indents, holes, perforations, reliefs and scores.

5. The device of claim 1, wherein the housing unit further comprises a cap positioned at the distal end within the interior enclosure, wherein the cap is configured to attach to the inlet port and block access to the catheter line.

6. The device of claim 5, wherein the cap comprises a base and sidewalls extending up proximally from said base forming a cavity region with a rim.

7. The device of claim 6, further comprising a sealing member connected to the rim and sealing the cavity region of the cap.

8. The device of claim 7, wherein the sealing member is selected from the group consisting of: cellophane, PVC (Polyvinylidene Chloride), LDPE (Low-Density Polyethylene), polypropylene, foil, and antiseptic impregnated material.

9. The device of claim 5, further comprising one or more antiseptic fluids held within the cavity region of the circular container.

10. The device of claim 9, wherein the antiseptic fluids is selected from the group consisting of: isopropyl alcohol, isopropanol, chlorhexidine, povidone-iodine, chloroxylenol, hexachlorophene, benzalkonium chloride, and hydrogen peroxide.

13. A device for prevention of vascular access port tampering comprising:
a housing unit having a proximal end, a distal end and an interior enclosure configured to receive a catheter line inlet port thereinto;
an opening in the proximal end of the housing unit, passing through said housing unit to the interior enclosure, and forming a peripheral rim on the proximal end of the housing unit configured to allow passage of tubes attached to the inlet port; and
wherein the opening is formed with one or more separable proximal leaflets in the proximal end of the housing unit configurable from a first open position to a second closed position.

14. The device of claim 13, wherein the housing unit further comprises a cap positioned at the distal end within the interior enclosure, wherein the cap is configured to attach to the inlet port and block access to the catheter line.

15. The device of claim 14, wherein the cap comprises a base and sidewalls extending up proximally from said base forming a cavity region with a rim.

16. The device of claim 15, further comprising a sealing member connected to the rim and sealing the cavity region of the cap.
